(19) United States
(12) Patent Application Publication
(10) Pub. No.: US 2007/0009083 A1
(43) Pub. Date: Jan. 11, 2007

(54) System and method of an improved X-ray imaging detector

(76) Inventors: Jian-Qiang Liu, Campbell, CA (US); Xiao-Dong Sun, Fremont, CA (US); Jian-ying Cao, San Jose, CA (US)

Correspondence Address: SHERR & NOURSE, PLLC, 620 HERNDON PARKWAY, SUITE 200, HERNDON, VA, 20170, US

(21) Appl. No.: 11/077,560

(22) Filed: Mar. 10, 2005

Related U.S. Application Data
(60) Provisional application No. 60/552,413, filed on Mar. 10, 2004.

(51) Int. Cl. A61B 6/14

(52) U.S. Cl. 378/38

(57) ABSTRACT

System and method of a compact X-ray imaging detector for dental imaging applications are disclosed. In one of the preferred embodiments, a compact X-ray point source is placed in patient's mouth whereas a X-ray detector is placed outside. The preferred improvement offers several advantages over prior art practices. One of the advantages is the much-reduced X-ray exposure to only areas to be imaged. An additional advantage is the improvement on image resolution due to geometric magnification associated with the location of the detector. The third substantial improvement is the enhanced contrast due to reduction of off-axis scattering. In another preferred embodiment, an X-ray imaging detection system is disclosed. The X-ray imaging system consists of a compact X-ray source that can be placed into a patient's mouth, an optical detector with scintillator, system control and an interfaced computer.

This application claims priority to the provisional application entitled “Advanced X-ray Digital Dental Imaging System”, Ser. No. 60/552,413, filed by the same subject inventors and assignee as the subject invention on Mar. 10, 2004.

### BACKGROUND OF THE INVENTION

1. Field of the Invention

The present invention relates generally to the field of X-ray imaging and more particularly to a system and a method for imaging human teeth for dental applications.

2. Background Art

Over the last several years, digital X-ray detector based dental imaging systems are being developed due to much better dynamic range and detection quantum efficiency of these detectors in comparison with conventional X-ray films. Schick et al disclosed a typical prior art system in U.S. Pat. No. 5,995,583 (issued on Nov. 30, 1999) and this patent is therefore incorporated herein by reference as relevant background material. The prior art system consists of a digital X-ray detector 101, patient's teeth 102, X-ray beam 103, and X-ray source 104, as illustrated in FIG. 1. As can be observed from FIG. 1, in the conventional dental X-ray protocol, the X-ray source is placed outside the patient's mouth, while an X-ray sensor or film is placed into patient's mouth to obtain dental X-ray imaging.

There are several areas of the prior art system that can be improved. For instance, it is desirable to reduce the X-ray dosage for heath considerations. It is also desirable to have a portable system such that the system can be carried to areas without a dental office. A portable X-ray imaging system may find other applications in areas such as medical, military, and security. There is a need therefore for improved art such that a potable and compact X-ray imaging system can be assembled.

### SUMMARY OF THE INVENTION

The present invention discloses a method and a system related to portable digital X-ray imaging devices. At the center of these systems are miniature X-ray sources that can be inserted directly into a patient's mouth. These imaging systems also employ digital X-ray imaging detectors, instead of conventional films. The disclosed preferred geometry of X-ray source-object (teeth)-imaging detector provides substantial enhancements of system performance such as lower X-ray dosage, higher image resolution and contrast.

### BRIEF DESCRIPTION OF THE DRAWINGS

The aforementioned objects and advantages of the present invention, as well as additional objects and advantages thereof, will be more fully understood hereinafter as a result of a detailed description of a preferred embodiment when taken in conjunction with the following drawings in which:

FIG. 1 illustrates a prior art X-ray imaging system;

FIG. 2 shows the layout of an improved system incorporating a small X-ray source;

FIG. 3 displays a more detailed structure of the improved system;

FIG. 4 illustrates the cross-section of the portable X-ray source with a pyroelectric crystal, a shutter and a metaltarget;

FIG. 5 illustrates a compact X-ray source with a pyroelectric crystal, a shutter and a fiber capillary optics.

### DETAILED DESCRIPTION OF THE INVENTION

The present invention discloses a method and a system related to portable digital X-ray imaging devices. At the center of these systems are miniature X-ray sources that can be inserted directly into a patient's mouth. These imaging systems also employ digital X-ray imaging detectors, instead of conventional films. The preferred geometry disclosed herein of X-ray source-object (teeth)-imaging detector provides substantial enhancements of system performance such as lower X-ray dosage, higher image resolution and contrast.

Improved System Layout, Source-Object-Detector Geometry

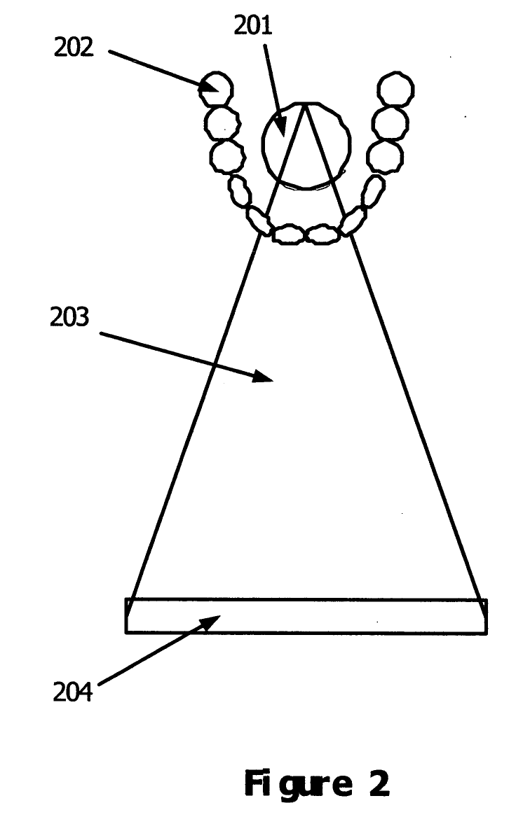

FIG. 2 displays one of the preferred embodiments of the present invention. An improved digital X-ray imaging system consists of a miniature X-ray source (201) being placed inside of a patient's mouth, and a digital or film X-ray detector (204) being placed at 1.5 to 5 times the distance from the object (202, teeth).

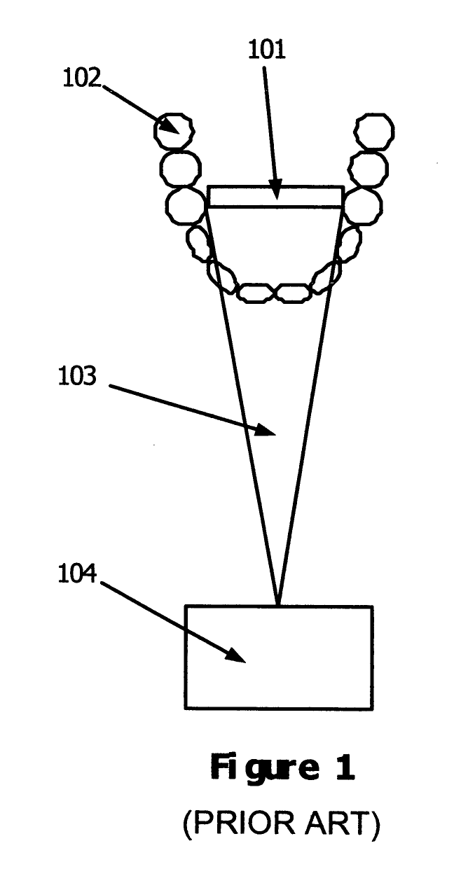

Contrast to prior art method as illustrated in FIG. 1, the improved art offers three major advantages: (1) Reduced X-ray exposure to other parts of the body; (2) Improved image resolution; and (3) Improved image contrast.

As can be visualized from FIGS. 1 and 2, in the prior art layout, since the light source is being placed outside of the patient's mouth, there are undesirable X-ray irradiation on other parts of human head (103) and frequently X-ray stoppers have to be applied to block some of them from X-ray exposure. With the improved layout, miniature point source X-ray (201) is being placed inside patient's mouth in close vicinity only to areas of objects (teeth). The expanded X-ray (203) escapes into the air without irradiating other regions of human head.

Another improvement is enhanced image resolution. There is an X-ray “magnifying” effect in the preferred layout. As illustrated in FIG. 2, since the source to detector distance is longer than source to object (teeth) distance, the X-ray image recorded is in effect larger than the object (teeth). This magnification results in a significantly improved spatial resolution (e.g. 1.5×-5×).

An additional improvement is enhanced X-ray image contrast: The interaction of X-ray with teeth will induce undesirable scattering into different angles that lower the image contrast. In the improved layout, the scattering beams became less likely to strike the detector thereby enhances image contrast.

Details of the Detection System

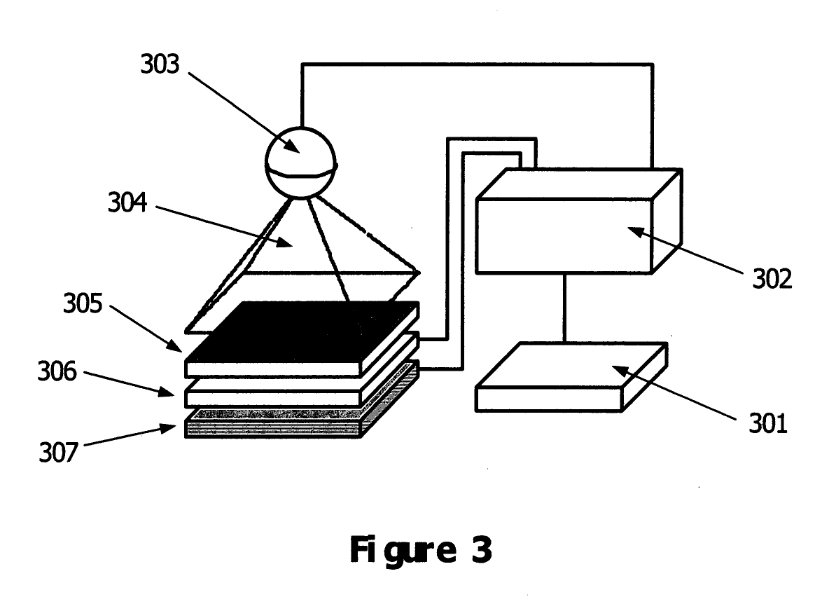

As displayed in FIG. 3, the improved X-ray imaging system consists of the following parts: A computer (301) which processes digital images; Control electronics (302); A miniature X-ray source (303); An X-ray beams penetrating objects (304); A high resolution X-ray scintillator (305); An image sensor (306); and a TEC cooler (307). A gum-ball sized high performance X-ray point source can be used for such X-ray imaging system. The gumball sized X-ray source is placed inside of a patients mouth and the detector outside. The miniature X-ray source can be freely rotated in patient's mouth to selectively irradiate various dental regions without irradiating onto other parts of head. The X-ray source has a very wide window for the most efficient utilization of X-ray photons. The digital detector can be a large area (>2″×2″) CMOS detector with a proprietary scintillator plate design where various X-ray scintillators are used to fill in anti-scattering grids.

A critical factor affecting X-ray image quality is the existence of scattering X-ray from the objects into detector. To overcome this effect, a preferred high performance digital X-ray detector is designed using registered grid plate (with underlying detector pixel array) to eliminate the scattering X-ray from meeting detector elements. The application of the grid plate significantly reduces the X-ray scattering noise and cross-talking of pixels in detecting X-rays, and improves the performance of current digital X-ray imaging detectors. Advanced X-ray flat panel display (FPD) with high X-ray luminosity and Detector Quantum Efficiency (DQE), fast response, high modulation transfer function (MTF), and high ratio of direct-to-scattered X-rays can be achieved simultaneously without any compromise. The structure of these X-ray detectors has been disclosed in more detail in a pending U.S. patent application (application No. 10/866,408, filed on Jun. 12, 2004) and this application is included herein as further background materials.

Miniature X-Ray Source

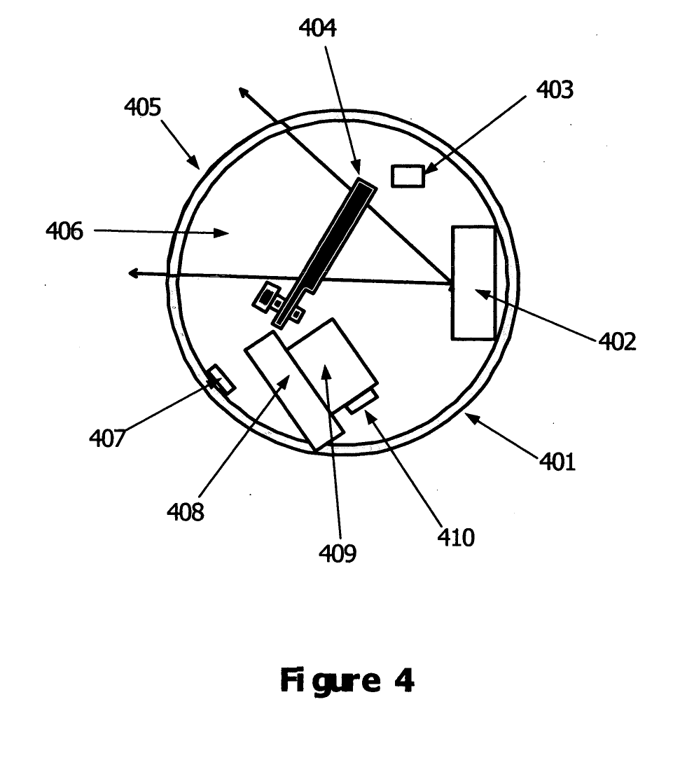

FIG. 4 shows a schematic of our disclosed gumball size X-ray source for the compact dental X-ray imaging system. The source integrates the pyroelectric crystal (409), temperature control (408), metal target (402), and X-ray dose control (404). The source fits into a round shaped package with maximum diameter less than 1.5 inches.

Our design of the gumball size X-ray source addresses many potential concerns related with the imaging quality, operation safety, convenience of changing the targeted teeth, and comfort. First the package uses a double-shell housing. Both the inner and outer shells (401) are made of heavy metal to absorb most unwanted X-rays. Cooling water runs between the shells to make the outer shell at comfort temperature for human mouth. A large and rectangle beryllium glass window (405) allows X-rays emitting from the target into a wide angle (˜20°×40°). A mechanical shutter (404) and a Si X-ray detector (403) form the feedback loop for X-ray dose control. The metal X-ray target (402) is firmly attached to the inner shell such that heat generated by the electron beam bombardment can be carried away quickly by cooling water. The pyroelectric crystal (409) attached to a high power TEC heater/cooler (408) sits on the opposite side of the metal target. The distance between the surface of the pyroelectric crystal to that of the metal target is such that self focused electron beam hits the metal at its smallest spot-size. Several thermal sensors (407, 410) are used to monitor the temperature of pyroelectric crystal (409), metal target (402) and metal shell (401) for reliable and safe operation of the X-ray source. A double shell package with cooling water flowing in-between the shells offers efficient cooling. Both shells are water and air leak-tight such that a low pressure (˜10 mtorr) is maintained inside for efficient X-ray generation.

One significant advantage of this system is that it can be operated at low vacuum (˜10 mtorr) instead of high vacuum required for conventional X-ray tubes. As a result, the cooling of the metal target and the system can be made much simpler than conventional high vacuum X-ray tubes. To increase X-ray flux, one can increase the surface area of the crystal and install an extra focusing mechanism to reduce the X-ray spot size.

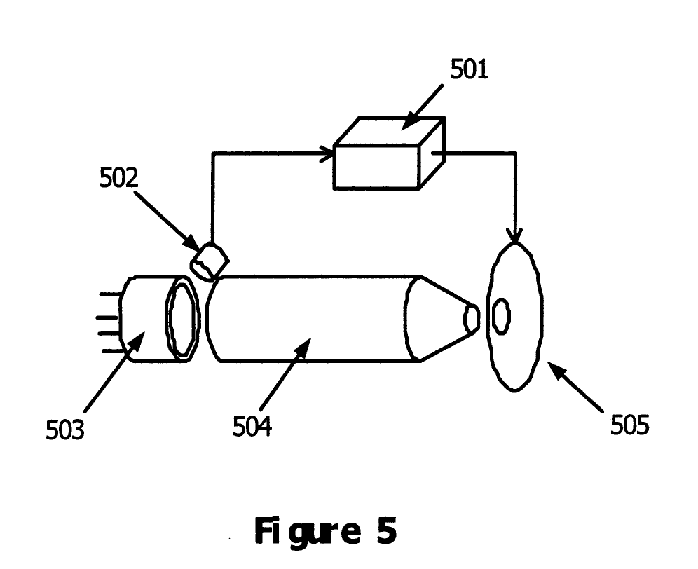

To meet the requirements of a clinical quality dental imaging system, X-ray source should output high-energy photons in the range of 30˜80 keV, and the output X-ray dose should be easily controllable and repeatable over time. As demonstrated in FIG. 5, a multi-fiber capillary (504) can be used with a miniature x-ray generator (503). Based on specification of the multi-fiber capillary, the spot size of this point X-ray source is around 15 μm (FWHM). In comparison, most X-ray tubes on the market have a spot size of about a few microns. Further reduction of spot size is possible through customer-specific optimization of the polycapillary. Nevertheless, at the size of 15 μm, the spot size of the X-ray source closely matches with Rad-icon CMOS X-ray detector which has a pixel size of ˜49.3×49.2 μm².

It will be apparent to those with ordinary skill of the art that many variations and modifications can be made to the system and method of portable digital X-ray imaging devices disclosed herein without departing form the spirit and scope of the present invention. It is therefore intended that the present invention cover the modifications and variations of this invention provided that they come within the scope of the appended claims and their equivalents, we claim:

1. A portable X-ray digital imaging system comprising:
a miniature X-ray source;
an X-ray image detector;
a controlling electronic circuitry for the said X-ray source and the said X-ray detector;
a computer being interfaced with the said X-ray source and the said X-ray image detector.

2. The X-ray imaging system recited in claim 1 wherein the said miniature X-ray source containing at least one pyroelectric crystal.

3. The X-ray imaging system recited in claim 2 wherein the said miniature X-ray source containing at least one Thermal Electric Cooler, or other heating and/or cooling element, attaching the said pyroelectric crystal.

4. The X-ray imaging system recited in claim 1 wherein the longest body diagonal of said miniature X-ray source is smaller than 3 inches.

5. The X-ray imaging system recited in claim 1 wherein the said miniature X-ray source containing at least one shutter.

6. The X-ray imaging system recited in claim 1 wherein the said miniature X-ray source being enclosed with a double layered metallic housing.

7. The X-ray imaging system recited in claim 6 wherein the said double layered metallic housing having cooling water stored between the said double layers.

8. The X-ray imaging system recited in claim 6 wherein the said double layered housing having a region of window material transmitting the said X-ray.

9. The X-ray imaging system recited in claim 1 wherein the said detector being a digital X-ray detector.

10. The X-ray imaging system recited in claim 1 wherein the said X-ray image detector containing an X-ray scintillator.

11. A method of recording digital X-ray image comprising the steps of:
placing a miniature X-ray source inside patients mouth;
illuminating object with the said X-ray source;
recording images with an X-ray image detector.

12. The method recited in claim 11 wherein the said miniature X-ray source containing at least one pyroelectric crystal.

13. The method recited in claim 12 wherein the said miniature X-ray source containing at least one Thermal Electric Cooler, or other heating and/or cooling element, attaching the said pyroelectric crystal.

14. The method recited in claim 11 wherein the said X-ray image detector contains a digital image sensor.

15. The method recited in claim 11 wherein the said X-ray image detector contains an X-ray film.

16. The method recited in claim 11 wherein the said miniature X-ray source being enclosed with a double layered metallic housing.

17. The method recited in claim 16 wherein the said double layered metallic housing having cooling water stored between the said double layers.

18. The method recited in claim 16 wherein the said double layered metallic housing having a region of window material transmitting the said X-ray.

19. The method recited in claim 11 wherein the said detector containing at least one anti-scattering grid.

20. The method recited in claim 11 wherein the said X-ray detector containing at least one X-ray scintillator.
